(19) United States
(12) Patent Application Publication
(10) Pub. No.: US 2012/0239119 A1
(43) Pub. Date: Sep. 20, 2012

(54) INFANT WARMER APPARATUS AND METHOD

(76) Inventors: Sreedhar Jyothigowdanapura Vyasarao, Bangalore (IN); Robin Mathew Issac, Bangalore (IN); Naresh Rajabhau Mungilwar, Pune (IN)

(21) Appl. No.: 13/422,612

(22) Filed: Mar. 16, 2012

(30) Foreign Application Priority Data
Mar. 17, 2011 (IN) 814/CHE/2011

(51) Int. Cl. A61N 5/06

(52) U.S. Cl. 607/88

(57) ABSTRACT

An infant warmer includes a LED driver, and a LED element operatively connected to the LED driver. The LED element has a characteristic spectral radiation wavelength range selected to optimize absorption by water molecules within an infant to thereby raise the infant's body temperature.

### BACKGROUND OF THE INVENTION

The subject matter disclosed herein relates to an infant warmer apparatus and associated method. More precisely, the subject matter relates to an infrared light emitting diode (LED) based infant warmer.

Warming devices may be implemented to warm an infant and to supply the necessary heat to maintain the infant within a predetermined temperature. Infant warmers commonly have an overhead radiant heater that is located above the infant and which thus radiates energy in the infrared spectrum to impinge upon the infant to maintain the predetermined temperature.

One problem with conventional infant warmers is that they have significant power requirements.

### BRIEF DESCRIPTION OF THE INVENTION

The above-mentioned shortcomings, disadvantages and problems are addressed herein which will be understood by reading and understanding the following specification.

In an embodiment, an infant warmer includes a LED driver, and a LED element operatively connected to the LED driver. The LED element has a characteristic spectral radiation wavelength range selected to optimize absorption by water molecules within an infant to thereby raise the infant's body temperature.

In another embodiment, an infant warmer includes a LED driver adapted to provide a generally constant current, and a controller operatively connected to the LED driver. The controller is configured to regulate operation of the LED driver. The infant warmer also includes a plurality of LED elements each operatively connected to the LED driver and adapted to receive the generally constant current. The LED elements have a characteristic spectral radiation wavelength in the range of 1400-1500 nm to optimize absorption by water molecules within an infant.

In another embodiment, a method for warming an infant includes providing an IR LED having a characteristic spectral radiation wavelength in the range of approximately 1400-1500 nm, and implementing the IR LED to emit electromagnetic radiation optimized for absorption by water molecules within an infant. The method for warming an infant also includes directing the electromagnetic radiation toward an infant such that the electromagnetic radiation is absorbed by the water molecules within the infant and the infant is thereby warmed.

Various other features, objects, and advantages of the invention will be made apparent to those skilled in the art from the accompanying drawings and detailed description thereof.

### BRIEF DESCRIPTION OF THE DRAWINGS

FIG. 1 is an isometric view of an infant warmer in accordance with an embodiment; and

FIG. 2 is a detailed schematic view of a plurality of light emitting diodes in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

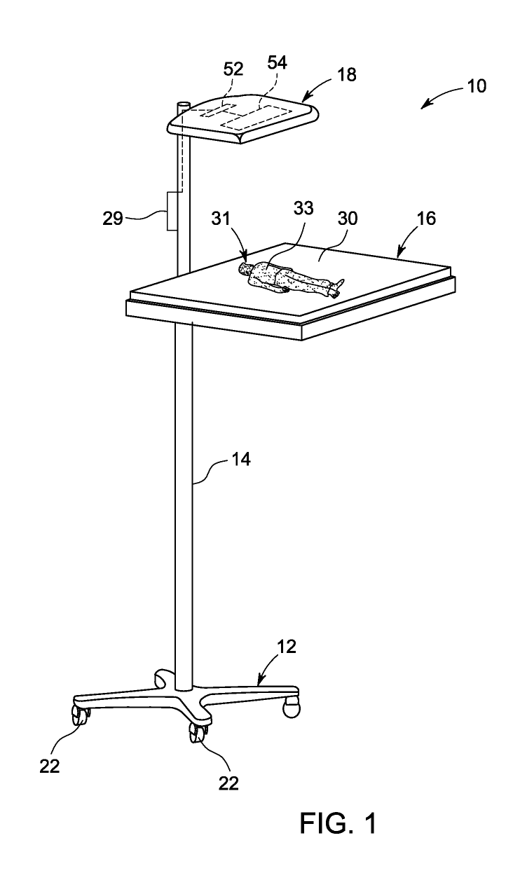

Referring to FIG. 1, an isometric illustration depicts an infant warmer 10 in accordance with one embodiment. The infant warmer 10 may include a base 12, a frame 14, a crib 16, and a heater assembly 18. It should be appreciated that the infant warmer 10 is being described in accordance with an embodiment, and that other configurations may be envisioned.

The base 12 may include a plurality of wheels 22 adapted to facilitate translation of the infant warmer 10. The frame 14 is secured to and extends away from the base 12 in a vertical direction. The frame 14 may optionally comprise a controller 29. The controller 29 is operable by a user to regulate the operation of the infant warmer 10.

According to the depicted embodiment, the crib 16 is mounted to the frame 14. The crib 16 includes a generally rectangular infant pedestal 30 that underlies and supports an infant 31. The infant pedestal 30 may comprise a padded mattress to comfort the infant 31. The infant 31 comprises a plurality of water molecules 33. The crib 16 may optionally include a plurality of walls (not shown) disposed on all four sides of the infant pedestal 30. The optional walls may be provided to retain the infant 31 within the infant warming apparatus 10.

The heater assembly 18 may be secured directly to the frame 14 as shown. The heater assembly 18 comprises a housing 50, a light emitting diode (LED) driver 52 disposed within the housing 50, and one or more LED elements 54 also disposed within the housing 50. The LED driver 52 is preferably coupled with the controller 29 such that a user can implement the controller 29 to regulate the operation of the LED driver 52. The LED driver 52 may be configured to provide a constant current source to power the LED elements 54. The LED elements 54 are each configured to receive current from the LED driver 52, and to emit electromagnetic radiation at a predefined spectral wavelength. The LED elements 54 are aligned with the crib 16 such that emitted electromagnetic radiation is directed toward the infant 31.

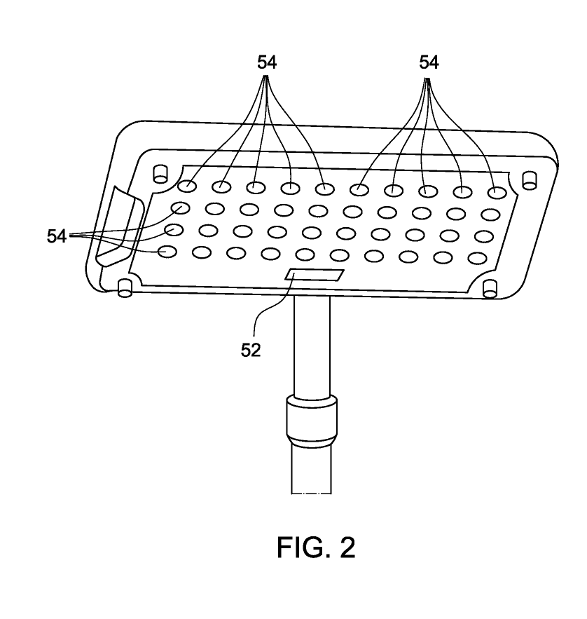

Referring now to FIG. 2, the LED driver 52 and the one or more LED elements 54 are shown in accordance with an embodiment. The LED elements 54 are each connected to and driven by the LED driver 52. The LED elements 54 may also be evenly distributed into a plurality of rows and columns to ensure the electromagnetic radiation emitted therefrom is uniformly dispersed over the target infant 31. As an example, the LED elements 54 may comprise four rows of ten Infrared (IR) LEDs. This geometric arrangement of LED elements has been shown to uniformly disperse emitted electromagnetic radiation across the surface of the pedestal 30 such that the infant 31 is uniformly treated regardless of relative position within the crib 16.

The LED elements 54 are preferably IR LED elements comprising a spectral radiation wavelength in the range of 1400-1500 nm, and most preferably of approximately 1450 nm. The preferred spectral radiation wavelength range of 1400-1500 nm is in the IR-B region of the full IR spectrum.

As the electromagnetic radiation from the LED elements 54 is transmitted through a medium containing water, the water molecules absorb portions of the electromagnetic spectrum. This water absorption occurs preferentially at wavelengths in the range of 1400-1500 nm. Accordingly, the spectral radiation wavelength range of the LED elements 54 has been specifically selected to optimize electromagnetic absorption by water molecules 33 within the infant 31. Electromagnetic absorption by water molecules 33 within the infant 31 has the effect of raising the infant's body temperature. It should therefore be appreciated that the heater assembly 18 and the LED elements 54 are particularly well suited to warming the infant 31.

Advantageously, the infant warmer 10 comprising LED elements 54 requires approximately 1/20^{th} as much power to operate compared to infant warmers with conventional heating elements. This reduced power consumption is particularly significant in developing countries in which traditional power sources may less reliable.

According to an embodiment, each of the LED elements 54 may be capable of producing 2 mW at the source. By implementing forty such identical LEDs as shown in the embodiment of FIG. 2, a total irradiance of 80 mW is produced at the source. The forty LED elements 54 may collectively be adapted to produce an irradiance level of 45 mW/cm² at a distance of 35 cm from the infant pedestal 30.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. An infant warmer comprising:
a LED driver; and
a LED element operatively connected to said LED driver, said LED element having a characteristic spectral radiation wavelength range selected to optimize absorption by water molecules within an infant to thereby raise the infant's body temperature.

2. The infant warmer of claim 1, wherein the LED element comprises a plurality of LED elements that are collectively adapted to produce an irradiance level of at least 80 mW at the source.

3. The infant warmer of claim 1, wherein the LED element comprises a plurality of LED elements that are distributed in a manner adapted to ensure that an emitted electromagnetic radiation is uniformly dispersed over the infant.

4. The infant warmer of claim 3, wherein the plurality of LED elements are evenly distributed into a series of rows and columns.

5. The infant warmer of claim 1, wherein the LED element comprises a characteristic spectral radiation wavelength in the range of 1400-1500 nm.

6. The infant warmer of claim 1, wherein the LED element comprises a characteristic spectral radiation wavelength of approximately 1450 nm.

7. An infant warmer comprising:
a LED driver adapted to provide a generally constant current;
a controller operatively connected to the LED driver, said controller configured to regulate operation of the LED driver; and
a plurality of LED elements each operatively connected to said LED driver and adapted to receive said generally constant current, said plurality of LED elements having a characteristic spectral radiation wavelength in the range of 1400-1500 nm to optimize absorption by water molecules within an infant.

8. The infant warmer of claim 7, wherein the plurality of LED elements are collectively adapted to produce an irradiance level of at least 80 mW at the source.

9. The infant warmer of claim 7, wherein the plurality of LED elements are distributed in a manner adapted to ensure that an emitted electromagnetic radiation is uniformly dispersed over the infant.

10. The infant warmer of claim 7, wherein the plurality of LED elements are evenly distributed into a series of rows and columns.

11. The infant warmer of claim 7, wherein one or more of the plurality of LED elements comprise a characteristic spectral radiation wavelength of approximately 1450 nm.

12. A method for warming an infant comprising:
providing an IR LED having a characteristic spectral radiation wavelength in the range of approximately 1400-1500 nm;
implementing the IR LED to emit electromagnetic radiation optimized for absorption by water molecules within an infant;
directing said electromagnetic radiation toward an infant such that said electromagnetic radiation is absorbed by the water molecules within the infant and the infant is thereby warmed.

13. The method of claim 12, wherein said providing an IR LED having a characteristic spectral radiation wavelength in the range of approximately 1400-1500 nm comprises providing an IR LED having a characteristic spectral radiation wavelength of approximately 1450 nm.

14. The method of claim 12, wherein said directing said electromagnetic radiation toward an infant comprises generally uniformly dispersing said electromagnetic radiation over the infant.
